(19) United States
(12) Patent Application Publication
(10) Pub. No.: US 2026/0151097 A1
(43) Pub. Date: Jun. 4, 2026

(54) COMPUTER-IMPLEMENTED METHOD FOR OPERATING AN X-RAY IMAGING DEVICE, X-RAY DEVICE, COMPUTER PROGRAM AND ELECTRONICALLY READABLE DATA STORAGE MEDIUM

(71) Applicant: Siemens Healthineers AG, Forchheim (DE)

(72) Inventors: Annette Birkhold, Stuttgart (DE); Philipp Roser, Allston, MA (US)

(21) Appl. No.: 19/408,335

(22) Filed: Dec. 3, 2025

(30) Foreign Application Priority Data
Dec. 4, 2024 (DE) 10 2024 211 574.6

(51) Int. Cl. A61B 6/04; G06T 7/73

(52) U.S. Cl. CPC A61B 6/0492; A61B 6/0407; G06T 7/75; G06T 2207/10116; G06T 2207/20021; G06T 2207/20081; G06T 2207/20084; G06T 2207/20092; G06T 2207/30088

(57) ABSTRACT

A method for operating an X-ray device includes ascertaining a patient model of a patient that describes a surface of the patient, based on patient information. A model position is determined for the patient model in a coordinate system of the X-ray device, in which radiation distribution during an X-ray image acquisition of the examination procedure is also known. The patient model is positioned in accordance with the model position. An X-ray image of the examination procedure, which is acquired for a current patient position of the patient in the X-ray device and exists in the coordinate system of the X-ray device, is supplied as input data to a trained position determination function. Output data from the trained position determination function describes a mapping of a distinguished point of the patient model to a distinguished point of the X-ray image. The model position is determined from the output data.

This application claims the benefit of German Patent Application No. DE 10 2024 211 574.6, filed on Dec. 4, 2024, which is hereby incorporated by reference in its entirety.

### BACKGROUND

The present embodiments relate to operating an X-ray imaging device.

Medical X-ray imaging devices are now commonplace in medical practice. Medical X-ray imaging devices use X-ray radiation that is emitted by an X-ray source and passes through an acquisition region of a patient in order to gain, using an X-ray detector, X-ray images that contain information about what is inside the patient. Since X-ray radiation may inherently have undesirable effects on humans, it is known in this context to monitor the X-ray dose to which the patient is exposed in an examination procedure involving, for example, multiple X-ray images to be acquired, and to provide corresponding dose information to an operator. Of particular interest here is what is known as the skin dose, which relates to regions along the surface of the patient and is specified according to spatial location. In order to allow accurate dose information relating to a patient to be determined, precise knowledge would be needed about the patient and his position with respect to the acquisition arrangement formed by X-ray source and X-ray detector (e.g., the position in the coordinate system of the X-ray device). This requires, however, complex additional sensor technology and/or additional X-ray acquisitions, which should ideally be avoided.

Therefore, it has been proposed with regard to obtaining dose information, but also for other usages, to use a patient model that may be patient-specific (e.g., a statistical shape model (SSM)). The patient model may describe the surface of the patient and is adapted using certain patient information (e.g., size, weight, and gender) sufficiently accurately to the actual patient. In addition to the use in dose observations, such virtual patient models may also be employed in many other areas of use (e.g., workflow automation, markerless tracking, positioning, and navigation assistance in minimally invasive image-guided interventions). For these reasons, provision may also be made to model in addition to the surface of the patient also other anatomic features (e.g., at least point-defined positions of internal organs and the like). For example, such patient models based on statistics are described in an article by Karthik Shetty et al., “BOSS: Bones, organs and skin-shape model,” Computers in Biology and Medicine 165 (2023 ) 107383. It is also possible to use artificial intelligence to adapt predefined patient models based on X-ray images of the patient; see, for example, the article by Karthik Shetty et al., “Deep Learning Compatible Differentiable X-Ray Projections for Inverse Rendering,” in: Palm, C., Deserno, T. M., Handels, H., Maier, A., Maier-Hein, K., Tolxdorff, T. (eds): “Bildverarbeitung für die Medizin 2021. Informatik aktuell.” [Image processing for medicine 2021. Current computer science], Springer Vieweg, Wiesbaden. [Proceedings, German Conference on Medical Image Computing] https://doi.org/10.1007/978-3-658-33198-6_70.

An existing problem with the use of patient models is ascertaining the most accurate possible positioning of the virtual patient model in the coordinate system of the X-ray imaging device in order to perform calculations using the model (e.g., to ascertain dose information). The aim is for the position of the patient model in a virtual calculation space to correspond as exactly as possible to the actual patient position adopted for the examination procedure. It is known to perform the positioning of the patient model in the coordinate system of the X-ray device based on user inputs (e.g., by a physician and/or an assistant medical technician). For example, it may be known for a certain procedure that the patient is normally positioned in the supine position with the head 10 cm below the end of the patient couch. It has been found, however, that in a large percentage of the specific examination procedures, the spatially resolved dose is calculated at the wrong location (e.g., the positioning assumption is incorrect or is at least not accurate enough).

It has also been proposed to register the patient model with acquired X-ray images, although to do this, the patient model is also to fully model the internal anatomy of the patient in order for the same structures to exist in the X-ray images and the patient model. Such patient models, which are complex, have a large data volume, and may be adapted only with difficulty to specific patients, are rarely employed in the prior art, however. Known patient models include only the surface of the patient (e.g., in a description as a polygon mesh) and possibly individual landmarks as points (e.g., points that represent in space the organs, such as centroid, distal end of an organ, proximal end of an organ, and the like). Classical registration methods cannot be applied here.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, more accurate positioning of a virtual patient model may be facilitated for an examination procedure in a coordinate system of the X-ray imaging device.

In a method according to the present embodiments, in order to ascertain a position of a patient model, at least one X-ray image of the examination procedure that is acquired for a current patient position of the patient in the X-ray device and exists in the coordinate system of the X-ray device, is supplied as input data to a trained position determination function. Output data from the trained position determination function describes a mapping of at least one distinguished point of the patient model to at least one distinguished point of the X-ray image. The model position is determined from the output data.

The patient model of the patient may be ascertained specifically, for example, using various methods known fundamentally in the prior art. In example embodiments, a statistical shape model (SSM) that is adapted based on the patient information. The patient information may include, for example, a size, weight, and/or gender of the patient. Also, further patient information that may be used to adapt the statistical shape model (e.g., age, build, and the like) may also be provided. It is also within the scope of the present embodiments, however, to ascertain the virtual patient model of the patient in other ways (e.g., from image data of an earlier examination procedure on the patient), in which, for example, the acquisition of the patient was at least partially in three dimensions and/or related to the surface of the patient. In all cases, however, the trained position determination function is obtained, such that the trained position determination function may be employed for all conceivable specific instances of the patient model (e.g., by having used training datasets for different instances of the patient model).

With regard to the problematic positioning of the patient model of the patient in the coordinate system of the X-ray device, it is provided to employ a trained position determination function (e.g., methods of artificial intelligence or machine learning). In this case, the trained position determination function is provided such that the trained position determination function is trained by machine learning to be able to ascertain from X-ray images of the patient at least approximately where on the patient one is located, so that the virtual patient model may be moved accordingly. Since X-ray images show an acquisition region of the patient, the X-ray images also contain information about which acquisition region is involved and where in the abstract this is located in the patient model. Also bearing in mind that the acquisition geometry is known in the coordinate system of the X-ray device, this yields at least in part the current patient position, to which the model position is meant to correspond as closely as possible.

If the coordinate system of the X-ray device relates to a patient couch, for example, which is provided as a patient support, it is possible to ascertain from the mapping of a point in the X-ray image to a point in the virtual patient model a coordinate on the patient couch (e.g., to which the virtual patient model (lying virtually on the virtual patient couch) is moved). Thus, a point of the virtual patient model (e.g., a point on the surface described by the virtual patient model) is mapped directly to a point in the X-ray image (e.g., its center or at least one of the corner points), thereby facilitating automatic positioning of the virtual patient model (e.g., a surface model) of the patient in the coordinate system of the X-ray device (e.g., taking into account the acquisition geometry known in the coordinate system of the X-ray device and at least one additional assumption, such that the patient is on the patient support). This saves time and avoids user mistakes that may lead to incorrect results from the functions using the virtual patient model. This requires no additional hardware (e.g., additional sensor technology such as cameras), because it works with X-ray images that are to be acquired anyway.

As already mentioned, using the procedure described is particularly expedient when direct registration approaches fail because the patient model does not provide the information needed for this. For example, a patient model that describes solely the surface of the patient (e.g., is a pure surface model) may be used. In one embodiment, the patient model describes, in addition to the skin surface, (e.g., as a single piece of additional information) at least one distinguished point of at least one organ. Thus, again in such a case, the patient model, for example, does not describe any bone and/or tissue structures that would allow direct registration. Yet, using the procedure according to the present embodiments, it is possible in this case to achieve accurate positioning in the coordinate system of the X-ray device.

The patient model may be used in a variety of ways (e.g., employed for different functions for calculating a result, such as for workflow automation or for obtaining dose information). It may be provided specifically that the dose information is calculated as a spatially resolved radiation effect on the positioned patient model (e.g., as skin dose information). As a result of the automated and, for example, robust positioning of the virtual patient model in the coordinate system of the X-ray device, the dose information may be obtained with sufficient accuracy in the correct position.

In general, a trained function models cognitive functions that humans associate with other human brains. As a result of training based on training data (e.g., machine learning), the trained function is capable of adapting to new circumstances and detecting and extrapolating patterns. Another expression for “trained function” is “trained machine learning model.”

Generally speaking, parameters of a trained function may be adapted by training. For example, supervised learning, semi-supervised learning, unsupervised learning, reinforcement learning, and/or active learning may be used. Further, representation learning (e.g., also known as feature learning) may also be employed. The parameters of the trained function may be adapted, for example, iteratively by a plurality of training steps. For example, a specific cost function may be minimized in the training. For example, the backpropagation algorithm may be employed in the training of a neural network.

A trained function may include, for example, a neural network, a support vector machine (SVM), a decision tree, and/or a Bayes network, and/or the trained function may be based on k-means clustering, Q-learning, genetic algorithms, and/or association rules. For example, a neural network may be a deep neural network, a convolutional neural network (CNN), or a deep CNN. In addition, the neural network may be an adversarial network, a deep adversarial network, and/or a generative adversarial network (GAN).

A convolutional neural network (CNN) is a neural network that uses a convolution operation instead of general matrix multiplication in at least one of its layers, known as the convolutional layer. For example, a convolutional layer performs a scalar product of one or more convolution kernels with the incoming data/images of the convolutional layer, where the entries in the one or more convolution kernels are the parameters or weights that are adapted by training. For example, the Frobenius inner product and the ReLU activation function may be used. A CNN may include additional layers (e.g., pooling layers, fully connected layers, and normalization layers).

Input images may be processed extremely efficiently by a CNN because a convolution operation based on different kernels may extract an extremely wide range of image features, and therefore, the relevant image features may be identified during the training by adapting the weights of the convolution kernel. Further, based on the sharing of the weights in the convolutional layer kernels, fewer parameters are to be trained, which prevents overfitting in the training phase and allows faster training or a larger number of layers in the CNN, thereby increasing the performance of the network.

In the present case, it may be provided, for example, that the trained position determination function includes a convolutional neural network and/or an encoder. An encoder analyzes the X-ray image and outputs the relevant features (e.g., the modeled region with regard to the patient model) as a low-dimensional feature vector, which, if applicable, a decoder may still decode from a latent space. It has been found that even simple generally known architectures are sufficient for achieving an excellent mapping quality.

One development of the present embodiments may provide that at least one of the at least one distinguished points of the X-ray image is the center point thereof. The center point of the X-ray image describes the attenuation experienced along the central ray of the X-ray field used. Since the acquisition geometry of the X-ray image is known in the coordinate system of the X-ray device, and hence also the location of the central ray, and since the patient may be assumed to be situated on the patient support of the X-ray device (e.g., on the patient couch), simple positioning is therefore possible based on the mapping of a point in the virtual patient model to the center point of the X-ray image. The virtual patient model is assumed, for example, to be positioned on a virtual model of the patient support (e.g., the patient couch). It may therefore be provided that the patient model is positioned such that the central ray of the X-ray field in the acquisition of the X-ray image used as input data, the position of which is known in the coordinate system of the X-ray device, passes through the distinguished point of the patient model when the patient model is in position on a patient support (e.g., a patient couch) of the X-ray device.

In the positioning of the patient model at the model position in the coordinate system of the X-ray device, orientation information that is available in association with the examination procedure and/or may be derived from a user input and/or may be ascertained from the X-ray image may additionally be used. For example, when positioning solely based on the central ray, additional information about the orientation of the patient on the patient support is to be provided at least when different orientations may be provided. Such orientation information may be available anyway (e.g., associated with a class of the examination procedure), since the same orientations may be used for certain classes of examinations. Such orientation information may also be derived from a user input and/or from the X-ray image itself (e.g., automatically). The orientation information may be kept simple (e.g., in the case of a patient couch, the orientation information specifies the end at which the head is positioned and also whether the supine position or prone position is adopted). In principle, using sensor technology of the X-ray device to obtain the orientation information is conceivable but less preferable because the procedure described here may be used specifically to avoid such additional sensor technology.

A development of the present embodiments may provide that the patient model describes the surface of the patient by polygons using vertices of the polygon faces, where the distinguished point of the X-ray image is mapped to a vertex of the patient model. The vertex, for example, is associated with the side of the skin surface facing the X-ray source of the X-ray device, or to an overlying vertex position ascertained from a plurality of adjacent vertices, as the distinguished point of the patient model. The surface of the patient is described in the patient model therefore as a polygon mesh containing corresponding vertices. In one embodiment, the trained position determination function may ascertain, for example, the nearest vertex to the corresponding point of the X-ray image, since the vertices represent clearly defined points in the patient model. For example, the vertices in the polygon mesh describing the surface may be numbered or otherwise labeled. In one embodiment, however, an overlying vertex position may be used as the distinguished point of the patient model from, for example, a plurality of vertices. It may be provided in this case, for example, that the vertex position is ascertained as the center point of a plurality of vertices mapped, for example, to an anatomical feature and/or an anatomically defined segment of the surface of the patient (e.g., a back portion). The center point may, for example, be constrained to lie on the surface. A vertex position may be used when rough positioning of the acquired body region is sufficient for the purposes of using the virtual patient model. Thus, for example, a plurality of vertices may be aggregated (e.g., vertices of the left hand and of the right hand and also of further anatomical features or limb portions). This may save training and computational effort relating to the trained position determination function, and make this function more robust. As regards choosing the side of the patient, if necessary, the orientation information may again be taken into account in both cases.

For example, in order to describe the surface of the patient in the patient model, a polygon mesh that has one to three thousand vertices (e.g., two thousand vertices) that may be spaced at distances of 1.5 cm and less, for example, may be used.

A development of the present embodiments provides that the X-ray image used as the input data is partitioned into sub-images, where the trained position ascertainment function is applied separately to each of the sub-images in order to obtain sub-results for each of the sub-images. In order to increase the accuracy, a current X-ray image may thus be partitioned into a plurality of evaluation regions (e.g., the sub-images), and output data (e.g., a reference vertex) may be determined for each region of the plurality of evaluation regions. Thus, the sub-images are evaluated individually (e.g., each are supplied to the trained position determination function as a separate input dataset), so that independent output datasets are attained as the sub-results. A development may then provide that the sub-results are checked against each other for consistency (e.g., by comparing the points in the patient model that are output for adjacent sub-images) in order to detect outliers. Detected outliers are excluded from the ascertaining of the model position. For example, the proximity in the patient model between all the determined distinguished points (e.g., vertices) may be assessed in order to check the consistency. Outliers may hence be detected robustly and ignored for the positioning of the virtual patient model.

Specifically, in order to partition the X-ray image into sub-images, it may be provided, for example, that a regular grid, or a grid adapted in terms of perspective according to the acquisition geometry of the X-ray image, is placed over the X-ray image. In the case of a grid for partitioning that is adapted to the perspective distortion caused by the acquisition geometry, it may be provided, for example, that outer sub-images are larger or else smaller than inner sub-images.

In one embodiment, the trained position ascertainment function is applied for a plurality of different partitionings of the X-ray image (e.g., including being applied to the entire X-ray image). For example, it may thus be provided that the sub-results from different partitionings are checked against each other for plausibility. One partitioning, for example, relates to a single sub-image (e.g., the use of the entire X-ray image as the input data to the trained position determination function).

The variant in which output data is obtained for different partitionings may be understood as an iterative hierarchical adaptation of the sub-images/evaluation regions. For example, the X-ray image may first be partitioned into small sub-images, which in subsequent iteration steps are iteratively aggregated and re-evaluated. Regardless thereof, even in the simplest implementation, partitioning into a plurality of sub-images and applying to the entire X-ray image, global information and local information may be gained and may be taken into account.

Further, the accuracy of the procedure according to the present embodiments may also be increased by using a plurality of X-ray images as (e.g., separate) input datasets in order to perform a plurality of determinations, the results of which may be combined by statistical processing and/or checked for plausibility. The X-ray image used may be at least one first X-ray image of the examination procedure. For example, a certain number of first X-ray images acquired in the examination procedure may be used.

A development of the present embodiments may provide that a determination of the model position carried out at a first point in time is verified and/or updated at at least one later point in time during the examination procedure by re-applying the trained position determination function to at least one current X-ray image. For example, it may be provided that the model position is determined every 30 seconds to 5 minutes. In one embodiment, for example, this may be provided when the acquisition of the X-ray images is used for image monitoring of a medical intervention (e.g., a minimally invasive intervention), which may extend over a prolonged period of time. Further, this may also be provided precisely in such cases to gain reliable spatially resolved dose information in order to adapt, if applicable, the acquisition geometry for the X-ray images, which then may be, for example, fluoroscopic images.

In one embodiment relating to the learning by the position determination function, in order to train the position determination function, X-ray images used as the training input datasets are obtained by simulation under a known relative positioning of the patient model, which describes associated training output datasets, and the position determination function is trained by the training datasets formed by associated training input datasets and training output datasets.

X-ray images simulated, for example, with different adapted or ascertained instances of the patient model have the advantage that the relative positioning and hence the output data are known in advance. Specifically, the simulation may include a Monte Carlo simulation and/or the deployment of a trained generating function for simulated X-ray images. For example, in the case of Monte Carlo simulation, attenuations based on simulated organ densities may be used, which may be assumed to be homogeneous, for example. Also, virtual X-ray images based on machine learning, which may be produced by deploying a suitable generating function, may be used to produce training datasets.

In the context of the disclosure described here, a computer-implemented method for providing a trained position determination function may be provided. The trained position determination function obtains, from input data that includes an X-ray image of a patient that exists in a coordinate system of an X-ray device that was used for its acquisition, output data that describes a mapping of at least one distinguished point of a virtual patient model of the patient that describes at least the surface of the patient, to at least one distinguished point of the X-ray image. X-ray images to be used as the training input datasets are obtained by simulation under a known relative positioning of the patient model that describes associated training output datasets. The position determination function is trained using the training datasets formed by associated training input datasets and training output datasets, and the trained position determination function is provided.

A corresponding provision system may be configured to perform this provision method. In one embodiment, a provision computer program that, when executed on a computing device, causes the computing device to perform the acts of the provision method is provided. In another embodiment, an electronically readable provision data storage medium (e.g., a non-transitory computer-readable storage medium), on which the provision computer program is stored, is provided.

In addition to the operating method, the present embodiments also relate to an X-ray imaging device having an X-ray source, an X-ray detector, a patient support (e.g., a patient couch) for supporting a patient to be examined in an examination procedure, and a control device. The control device includes a model ascertainment unit for ascertaining a virtual patient model of the patient that describes at least the surface of the patient, based on patient information describing the patient. The control device also includes a determination unit for ascertaining a model position for the patient model in a coordinate system of the X-ray device in which the radiation distribution during an X-ray image acquisition of the examination procedure is also known. The control device also includes a positioning unit for positioning the patient model in accordance with the model position.

The determination unit has: an application sub-unit for applying a trained position determination function to input data that includes at least one X-ray image of the examination procedure that is acquired for a current patient position of the patient in the X-ray device and exists in the coordinate system of the X-ray device, where the output data from the position determination function describes a mapping of at least one distinguished point of the patient model to at least one distinguished point of the X-ray image; and a model position sub-unit for determining the model position from the output data of the position determination function.

All the statements relating to the method according to the present embodiments may be applied analogously to the X-ray imaging device according to the present embodiments, and vice versa, and therefore the aforementioned advantages may likewise be achieved by the X-ray device.

The control device may include at least one processor and at least one storage device. The control device is configured, for example, to execute an operating method according to the present embodiments. Functional units are formed by hardware and/or software in order to implement acts of the operating method according to the present embodiments. In addition to the functional units already mentioned, further functional units may also be provided in order to execute further acts (e.g., example embodiments). For example, a training unit that may include a simulation sub-unit, for training the position determination function may be provided.

The control device may also have functional units that provide functions that use the positioned virtual patient model. For example, a dose ascertainment unit may be provided for obtaining dose information calculated as a spatially resolved radiation effect on the positioned patient model (e.g., as skin dose information). In addition, a workflow unit may be provided and may use the positioned patient model. Further functional units of the control device may also include, for example, an acquisition unit that controls the acquisition of the at least one X-ray image and/or further X-ray images of the examination procedure. A display device (e.g., a monitor) may be used to output X-ray images and information. For example, dose information (e.g., about the skin dose) may be output by such a display device (e.g., controlled by a user interaction unit of the control device).

A computer program according to the present embodiments may be loaded directly into a storage device (e.g., a memory) of a control device of an X-ray device, and includes program means, such that when the computer program is executed in the control device, the program means causes the control device to perform the acts of a method according to the present embodiments. The computer program may be stored on an electronically readable data storage medium (e.g., a non-transitory computer-readable storage medium) according to the present embodiments. The electronically readable data storage medium therefore includes control information stored thereon that includes at least one computer program according to the present embodiments and is configured such that when the data storage medium is used in a control device of an X-ray device, the control device is configured to perform a method according to the present embodiments. The data storage medium may be a non-transient data storage medium (e.g., a CD-ROM).

### BRIEF DESCRIPTION OF THE DRAWINGS

Further advantages and details of the present invention are presented in the example embodiments described below and with reference to the drawings, in which:

FIG. 1 shows a flow diagram of an example embodiment of an operating method;

FIG. 2 shows a schematic representation of an interference process of a used trained position determination function;

FIG. 3 shows a schematic diagram relating to determining a model position from output data from the trained position determination function;

FIG. 4 shows a schematic representation illustrating a variant of the operating method according to the present embodiments;

FIG. 5 shows an X-ray device according to the present embodiments; and

FIG. 6 shows the functional design of a control device of the X-ray device.

### DETAILED DESCRIPTION

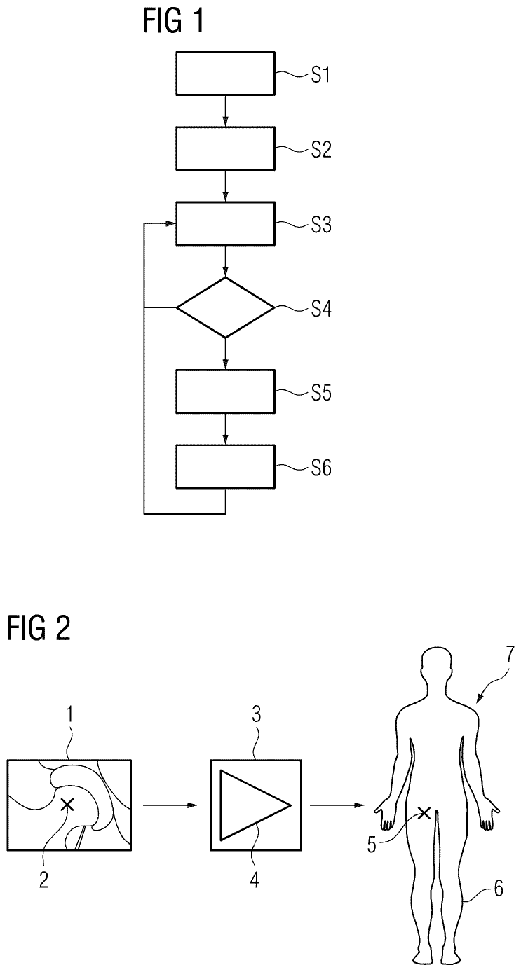

FIG. 1 shows a flow diagram of an example embodiment of an operating method for an X-ray imaging device. In act S1, an examination procedure on a patient begins, in the course of which a plurality of X-ray images are meant to be acquired by the X-ray imaging device in the present case. The patient is already positioned in a patient position for the acquisition of the X-ray images on a patient support device (e.g., a patient support) of the X-ray device (e.g., a patient couch). In addition, orientation information that describes how the patient is oriented (e.g., in the present case, at which end of the longitudinal extent of the patient couch the head is lying and whether the supine position or prone position is adopted) is already available. Also, patient information about the patient (e.g., including size, weight and gender) has already been captured and is available.

The examination procedure may involve, for example, image monitoring during a medical intervention (e.g., a minimally invasive intervention). In this process, fluoroscopic X-ray images are repeatedly acquired in order to represent visually the progress of the medical intervention.

In act S2, a virtual patient model for the patient is already ascertained. For example, the virtual patient model is ascertained by adapting a statistical shape model (SSM) based on the patient information. Other variants may also be provided (e.g., using previous image acquisitions of the patient). The patient model may also be ascertained so that the patient model is fully patient-specific.

In the present case, the patient model describes the surface of the patient as a polygon mesh having corresponding polygon faces and vertices. Optionally, the patient model may also describe distinguished points of organs, although the internal anatomic structure is not modeled in full, and therefore registration with acquired X-ray images is not possible.

In act S3, the first X-ray image of the examination procedure is then acquired.

In act S4, a check is carried out to determine whether a condition for positioning the patient model in a coordinate system of the X-ray device is satisfied. The coordinate system of the X-ray device may be defined with respect to the patient couch, for example. For acquired X-ray images, the acquisition geometry, and therefore also the location of the X-ray field, is inherently known in this coordinate system of the X-ray device. This provides that the X-ray image exists in the coordinate system of the X-ray device in the sense that for each pixel, the path of the corresponding beam along which the attenuation was measured is known.

In act S4, for the first positioning of the patient model in the coordinate system of the X-ray device, it may be sufficient that a first X-ray image of the examination procedure has been acquired; it is also possible, however, that a certain number of first X-ray images of the examination procedure are meant to be available. In the example embodiment shown, the positioning is checked regularly, which provides the condition in act S4 is also satisfied when, for example, a certain time has passed since the last positioning, which may be set between 30 seconds and 5 minutes, for example.

In act S5, if the condition in act S4 is satisfied, a trained position determination function is deployed. The trained position determination function uses input data that, in each case, includes one of the at least one first, or at a later point in time at least one of the at least one current, X-ray images to be used. The trained position determination function delivers output data that maps a distinguished point of the surface of the patient in the patient model onto a distinguished point of the X-ray image.

In the present case, the trained position determination function maps a vertex of the polygon mesh modeling the surface onto the center point of the X-ray image used as the input data, as is shown schematically by FIG. 2. This shows schematically first as input data the X-ray image 1 having the center point 2. The X-ray image 1 is supplied as input data to the trained position determination function 3. The trained position determination function 3, which is configured as at least one CNN, includes in the present case an encoder 4 that delivers results describing the corresponding vertex 5 of the surface 6 (e.g., indicated schematically) of the patient in the patient model 7. The vertex 5, located on the side at which the X-ray radiation enters, that is closest to the central ray is selected, for example (e.g., also taking into account the orientation information).

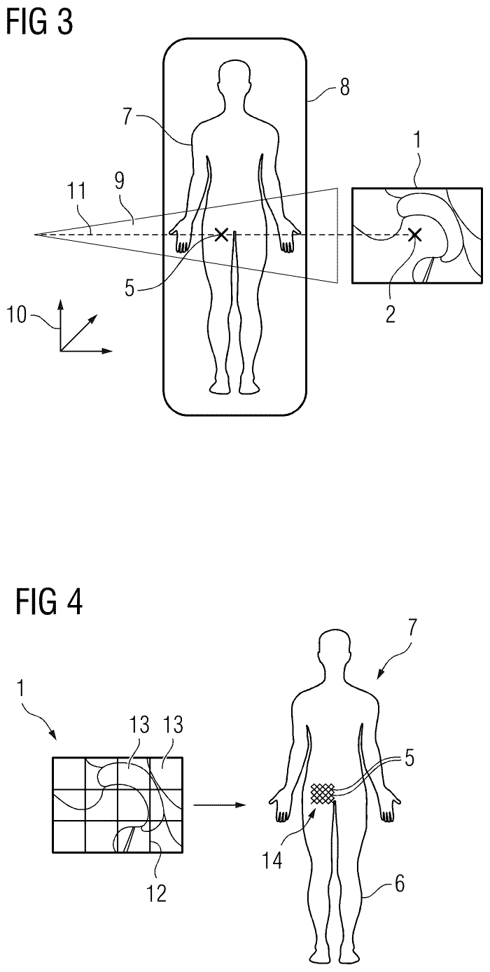

As FIG. 3 shows, this, together with the assumption that the patient is lying on the patient couch 8 (e.g., also indicated schematically) and the orientation information (e.g., supine position, head first), yields a suitable model position of the patient model 7, because the acquisition geometry and hence the X-ray field 9 used for the acquisition are known in the coordinate system 10 of the X-ray device. This applies, for example, to the central ray 11, along which the image value of the center point 2 of the X-ray image 1 was acquired, and which, according to the output data, is to travel through the vertex 5 described by this output data, as illustrated graphically in FIG. 3. Hence, the model position may be deduced, in act S5, from the output data, in this case together with the assumption that the patient is lying on the patient couch 8 and/or with the orientation information. If any uncertainty still exists (e.g., because of an unfavorable orientation of the central ray), a central location on the patient couch is assumed.

Vertices 5 need not necessarily be used as the distinguished points of the surface 6 of the patient model 7, but instead, distinguished points (e.g., vertex positions) that aggregate a plurality of vertices 5 (e.g., corresponding to anatomical features such as hands, feet, left abdomen, and the like) may also be used. For example, the vertex position may be a center point of vertices 5 to be aggregated. This slightly reduces the accuracy of the positioning but increases the robustness.

Also, other, or a plurality of, distinguished points in the X-ray image (e.g., corner points) may be used additionally or alternatively. For example, this may also give at least some indication of the orientation.

FIG. 4 illustrates a variant that may be used additionally or alternatively in act S4. In one embodiment, the X-ray image 1 is partitioned into sub-images 13 (e.g., using a grid 12 placed thereover), and the trained position determination function 3 is applied to each of the sub-images separately. The result is accordingly a group 14 of vertices 5, the relative position of which to each other on the surface 6 (e.g., their relative proximity) may be used for a mutual plausibility check or detection of outliers. In one embodiment, in act S4, output data for a plurality of partitionings (e.g., for an application to the entire X-ray image 1 and at least one partitioning into a plurality of sub-images 13) may be obtained. A mutual plausibility check may also be performed between the partitionings.

Returning to FIG. 1, in act S6, the patient model 7 is then positioned in accordance with the model position in the coordinate system 10 of the X-ray device. The patient model 7 may now be used by at least one function of the X-ray device (e.g., to obtain spatially resolved dose information in prolonged examination procedures, such as a spatially resolved skin dose).

The position determination function 3 has been trained by machine learning before being provided. In this process, at least some of the training datasets have been gained by simulations, in which X-ray images to be used as training input data under known model positions of different instances of the patient model were gained by Monte Carlo simulation and/or using a trained generating function, which performs at least part of the simulation implicitly.

FIG. 5 shows a schematic diagram of an X-ray imaging device 15 according to the present embodiments. In the present case, the X-ray imaging device is an X-ray device 15 having a C-arm 16 (e.g., as may be employed in interventional environments). The X-ray device 15 includes an X-ray source 17 and an X-ray detector 18 (e.g., as an acquisition arrangement) that are attached opposite each other on the C-arm 16. The C-arm 16 is movable and may be brought into different positions relative to the patient couch 8 in order to set up different acquisition geometries.

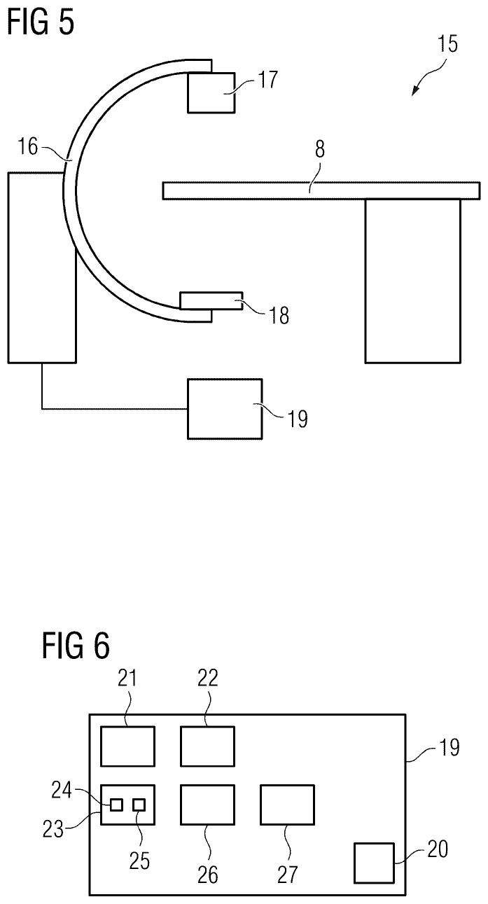

The operation of the X-ray device 15 is controlled by a control device 19, the functional design of which is described in greater detail with reference to FIG. 6. Accordingly, the control device 19 includes a storage device 20 (e.g., memory) for storing various information (e.g., the X-ray images 1, the orientation information, the patient information, the patient model 7, and the like).

A control unit, which is not shown in greater detail, controls the general operation (e.g., in accordance with acts S1 and S4). The control device 19 also has an acquisition unit 21 that controls the acquisition operation of the X-ray device 15. The control device 19 is also used to control the acquisition of X-ray images 1 in act S3. A model ascertainment unit 22 is configured to ascertain the virtual patient model 7 of the patient in accordance with act S2. In a determination unit 23, the model position for the patient model 7 is ascertained in accordance with act S5. For this purpose, the determination unit 23 has an application sub-unit 24 for applying the trained position determination function 3, and a model position sub-unit 25 for determining the model position from the output data of the position determination function 3 (e.g., as shown in FIG. 3). The control device 19 also includes a positioning unit 26 for positioning the patient model 7 in accordance with act S6.

With regard to the use of the positioned patient model 7, a dose unit 27 is also shown by way of example, in which the dose information, which gives a spatially resolved indication of the radiation dose acting on the patient, is obtained based on the positioned patient model 7 and the acquisition parameters of the X-ray images 1. For example, the dose information may be used for general monitoring (e.g., output of warnings if limit values are exceeded) and/or may be output on a display device of the X-ray device 15.

Further functional units may also be provided in addition to the functional units described (e.g., a user interaction unit and the like).

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for operating an X-ray imaging device, the method being computer-implemented and comprising:
providing a virtual patient model for an examination procedure on a patient, the providing of the virtual patient model comprising:
ascertaining the virtual patient model of the patient, which describes at least a surface of the patient, based on patient information describing the patient;
determining a model position for the virtual patient model in a coordinate system of the X-ray imaging device, in which coordinate system radiation distribution during an X-ray image acquisition of the examination procedure is also known; and
positioning the virtual patient model in accordance with the model position,
wherein determining the model position for the virtual patient model comprises:
supplying at least one X-ray image of the examination procedure that is acquired for a current patient position of the patient in the X-ray imaging device and exists in the coordinate system of the X-ray imaging device as input data to a trained position determination function, output data from which describes a mapping of at least one distinguished point of the patient model to at least one distinguished point of the at least one X-ray image; and
determining the model position from the output data.

2. The method of claim 1, wherein the virtual patient model describes, in addition to the surface of the patient, as a single piece of additional information, at least one distinguished point of at least one organ, and
wherein the surface of the patient is a skin surface.

3. The method of claim 1, wherein a distinguished point of the at least one distinguished point of the at least one X-ray image is a center point of the at least one X-ray image.

4. The method of claim 3, wherein the virtual patient model is positioned, such that a central ray of an X-ray field in the acquisition of the X-ray image used as input data, a position of which is known in the coordinate system of the X-ray imaging device, passes through the distinguished point of the patient model when the patient model is in position on a patient support of the X-ray imaging device.

5. The method of claim 1, wherein positioning the virtual patient model in accordance with the model position comprises positioning the virtual patient model at the model position in the coordinate system of the X-ray imaging device also using orientation information that is available in association with the examination procedure, is derivable from a user input, is ascertainable from the X-ray image, or any combination thereof.

6. The method of claim 1, wherein the virtual patient model describes the surface of the patient by polygons using vertices of polygon faces,
wherein a distinguished point of the at least one distinguished point of the X-ray image is mapped to a vertex of the virtual patient model, the vertex being associated with a side of the skin surface facing an X-ray source of the X-ray imaging device, or to an overlying vertex position ascertained from a plurality of adjacent vertices, as the distinguished point of the patient model.

7. The method of claim 1, wherein the X-ray image used as the input data is partitioned into sub-images, and
wherein the trained position ascertainment function is applied separately to each of the sub-images in order to obtain sub-results for each of the sub-images.

8. The method of claim 7, further comprising checking the sub-results against each other for consistency, the checking comprising comparing points in the virtual patient model that are output for adjacent sub-images of the sub-images, such that outliers are detected,
wherein the detected outliers are not used in the determining of the model position.

9. The method of claim 7, wherein the trained position ascertainment function is applied for a plurality of different partitionings of the X-ray image.

10. The method of claim 9, wherein the trained position ascertainment function is applied to the entire X-ray image.

11. The method of claim 1, wherein the trained position determination function comprises a convolutional neural network, an encoder, or the convolutional neural network and the encoder.

12. The method of claim 1, further comprising training the position determination function, training the position determination function comprising:
obtaining X-ray images to be used as training input datasets by simulation under a known relative positioning of the virtual patient model, which describes associated training output datasets; and
training the position determination function using training datasets formed by the training input datasets and the training output datasets.

13. The method of claim 12, wherein the simulation comprises a Monte Carlo simulation, deployment of a trained generating function for simulated X-ray images, or a combination thereof.

14. An X-ray imaging device comprising:
an X-ray source;
an X-ray detector;
a patient support configured to support a patient to be examined in an examination procedure; and
a control device comprising:
a model ascertainment unit configured to ascertain a virtual patient model of the patient that describes at least a surface of the patient based on patient information describing the patient;
a determination unit configured to ascertain a model position for the virtual patient model in a coordinate system of the X-ray imaging device in which radiation distribution during an X-ray image acquisition of the examination procedure is also known; and
a positioning unit configured to position the virtual patient model in accordance with the model position,
wherein the determination unit comprises:
an application sub-unit configured to apply a trained position determination function to input data that comprises at least one X-ray image of the examination procedure, the at least one X-ray image being acquired for a current patient position of the patient in the X-ray imaging device and existing in the coordinate system of the X-ray imaging device, wherein output data from the trained position determination function describes a mapping of at least one distinguished point of the virtual patient model to at least one distinguished point of the X-ray image; and
a model position sub-unit configured to determine the model position from the output data of the position determination function.

15. A non-transitory computer-readable storage medium that stores instructions executable by one or more processors to operate an X-ray imaging device, the instructions comprising:
providing a virtual patient model for an examination procedure on a patient, the providing of the virtual patient model comprising:
ascertaining the virtual patient model of the patient, which describes at least a surface of the patient, based on patient information describing the patient;
determining a model position for the virtual patient model in a coordinate system of the X-ray imaging device, in which coordinate system radiation distribution during an X-ray image acquisition of the examination procedure is also known; and
positioning the virtual patient model in accordance with the model position,
wherein determining the model position for the virtual patient model comprises:
supplying at least one X-ray image of the examination procedure that is acquired for a current patient position of the patient in the X-ray imaging device and exists in the coordinate system of the X-ray imaging device as input data to a trained position determination function, output data from which describes a mapping of at least one distinguished point of the patient model to at least one distinguished point of the X-ray image; and
determining the model position from the output data.
